(19) United States
(12) Patent Application Publication
(10) Pub. No.: US 2015/0125813 A1
(43) Pub. Date: May 7, 2015

(54) Topical Anesthetic Applicator, and Method for Numbing a Dental Area

(71) Applicant: Dentek Oral Care, Inc., Maryville, TN (US)

(72) Inventors: David A. Fox, Haverford, PA (US); Eric W. Mowell, Maryville, TN (US); Geoffrey A. de Rohan, Knoxville, TN (US); Charlotte E. Havely, Jefferson City, TN (US)

(73) Assignee: Dentek Oral Care, Inc., Maryville, TN (US)

(21) Appl. No.: 14/072,564

(22) Filed: Nov. 5, 2013

(51) Int. Cl. A61C 19/08; A61M 19/00

(52) U.S. Cl. CPC A61C 19/08; A61M 19/00; A61M 2210/0625
USPC 433/82; 433/215; 433/217.1; 604/290

(57) ABSTRACT

An oral care kit is provided. The oral care kit is designed to be used to treat a painful area of a patient's mouth, such as a tooth, an area of a lip, or an area along a gum line. The oral care kit generally includes a topical anesthetic, a plurality of disposable swabs, and an applicator. The applicator has a proximal end defining a handle, and a distal end comprising a pair of prongs. A channel is formed along an inner surface of the prongs. The proximal end of each swab is dimensioned to be slidably received within the channel, while the distal end comprises flocking. Each of the disposable swabs is dimensioned to be received within a user's mouth for application of the topical anesthetic. A method of numbing a dental area is also provided herein.

### CROSS REFERENCE TO RELATED APPLICATIONS

Not applicable.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### BACKGROUND OF THE INVENTION

1. Field of the Invention

The present invention relates to oral care devices. More specifically, the invention relates to an applicator used by an individual for applying a topical anesthetic to an area around the mouth or gums, and a method for temporarily numbing a dental area.

2. Technology in the Field of the Invention

Individuals occasionally suffer pain in the mouth and gums. Such pain may be due to the movement of teeth incident to the adjustment of braces or other orthodontic treatment. Such pain may alternatively be due to tooth development, or “teething,” in small children. Such pain may alternatively be due to irritation from dentures, a retainer, or other orthodontic appliances. Further, such pain may be due to something more serious such as tooth decay, i.e., loss of enamel, or nerve root irritation. Finally, an individual may suffer from a sore on the gums or lips.

In any of these instances, the individual may choose to apply a topical anesthetic to an affected area. Various ways for applying a topical oral anesthetic are known. First, it is known to apply a gel containing an anesthetic by using one's finger. In operation, a user will squeeze an amount of topical ointment from a tube and onto one's finger, and then rub the medicament onto an affected area.

In a more preferred manner, a swab such as the Q-tips® brand offered by Conopco, Inc. of Englewood Cliffs, New Jersey may be used. The swab comprises a wooden or plastic stick having a cotton or other open-celled absorbent material at opposing tips. In this instance, the anesthetic is squeezed or otherwise placed onto a cotton tip. The medicament is then applied using the swab.

More sophisticated types of swab applicators are known. U.S. Pat. No. 4,740,194 discloses a specially-design swab applicator wherein the applicator is in the form of a long, hollow tube. The tube is made up of a thin-walled plastic material. Within the tube is a liquid designed to be applied to an ear or other area of a patient. The liquid may be, for example, water, iodine or alcohol. A tab is provided proximate an end of the swab applicator. Removal of the tab allows the liquid held within the tube to be released, whereupon the liquid travels down to a tip of the swab for application.

In the context of oral gel applicators, such an applicator is offered by Blistex Inc. of Oak Brook, Illinois. This product is sold under the trade name Kank A®. The Kank A® product offers a tube holding an anesthetic liquid therein. The tube is generally shaped like a pen. At a tip of the tube is a brush. A user may swab an affected area in his or her mouth using the brush to apply an anesthetic.

Another product available on the market is the Orajel® cold sore treatment offered by Church & Dwight Co., Inc. of Ewing, New Jersey. In one embodiment, a vial is provided containing a topical anesthetic. The vial is snapped open, exposing an applicator tip. The vial is then held face-down to allow the tip to become saturated. The user may then apply the liquid anesthetic directly to an affected area such as a cold sore.

In another arrangement currently on the market, a small bottle is sold containing an oral topical anesthetic. The bottle is secured using a threaded lid. A stiff applicator is connected to the underside of the lid. The applicator is dipped into the bottle, removed from the bottle, and then rubbed onto the affected area.

Shortcomings exist with the known applicators. In some instances, the applicator requires use of an off-the-shelf swab or may involve use and multiple re-uses of the same plastic applicator. In other instances, the applicators require either that a tube or a vial be broken or a tab removed to expose a medicament. This increases complexity and cost of the device. Alternatively, the applicator may actually be a finger, meaning that a person's or parent's finger is placed into a mouth. This may not be the most sanitary approach. Accordingly, a need exists for an applicator for a topical anesthetic that permits the use of disposable and sterile swabbing tips.

### BRIEF SUMMARY OF THE INVENTION

An oral care kit is provided herein. The oral care kit is designed to be used to treat a painful area of a patient's mouth. Such an area may be, for example, a tooth, an area of a lip, or an area along a gum line. The patient may be an adult or an older child; alternatively, the patient may be an infant or a toddler.

In one embodiment, the oral care kit first includes a topical anesthetic. The topical anesthetic preferably comes in a tube or bottle having a lid that is threadedly secured to the tube or bottle. The topical anesthetic may be, for example, procaine, lidocaine, prilocaine, benzocaine, or combinations thereof.

The oral kit further comprises an applicator. The applicator includes a shaft. The shaft has a proximal end defining a handle for a user, and a distal end comprising a pair of prongs. A channel is formed along an inner surface of the prongs.

The applicator comes with a plurality of disposable swabs. Preferably, each swab is fabricated from a plastic material to form a flexible body that is between about 1 and 4 inches in length. Each swab comprises a proximal end and a distal end. The proximal end of the swab is dimensioned to be slidably received within the channel, while the distal end comprises hydrophilic flocking Preferably, the flocking comprises nylon and/or cotton fibers.

The applicator also includes a sleeve. The sleeve is slideably disposed along the shaft. The sleeve is movable from an open position that allows the prongs to open, thereby exposing the channel for receiving the proximal end of the swab, and a closed position wherein the channel closes around the proximal end of the swab to provide a friction fit. A shoulder may be provided along the shaft to limit travel of the sleeve towards the open position.

Each of the disposable swabs is dimensioned to be received within a user's mouth for application of the topical anesthetic. Preferably, the oral kit comprises a packaging containing the topical anesthetic and the applicator for retail sale. In one embodiment, the plurality of swabs comprises between about 10 and 50 individual swabs within the package.

A method of numbing a dental area is also provided herein. In one embodiment, the method first includes accessing an applicator. The applicator includes the shaft and the pair of prongs as described above. The applicator also includes the channel formed along an inner surface of the prongs, and a plurality of disposable swabs. Further, the applicator includes the sleeve slideably disposed along the shaft as described above.

The method also includes positioning the sleeve in its open position. The method then includes sliding the proximal end of a selected swab into the channel, and then moving the sleeve into its closed position. In this way, the selected swab is secured in place within the channel.

The method next includes unscrewing or otherwise removing a lid from the tube or bottle to access the topical anesthetic. The anesthetic preferably resides within a tube or a bottle. The method then includes applying a topical anesthetic to the distal end of the swab. In one aspect, the distal end of the swab is dipped into the tube or bottle.

The method also includes applying the topical anesthetic to an affected area of a patient's mouth. This is done by manually extending the swab into the patient's mouth to access the affected area, or around the lips. Thereafter, the disposable swab may be released from the prongs by sliding the sleeve back to its open position. A new swab may be selected later for a subsequent treatment to the same or a different affected area.

### BRIEF DESCRIPTION OF THE DRAWINGS

So that the manner in which the present invention can be better understood, certain illustrations, charts and/or flow charts are appended hereto. It is to be noted, however, that the drawings illustrate only selected embodiments of the inventions and are therefore not to be considered limiting of scope, for the inventions may admit to other equally effective embodiments and applications.

FIG. 1 is a perspective view of an applicator as may be used in an oral care kit of the present invention, in one embodiment. In this view, a swab is connected to a lip of the applicator.

FIG. 2A is an enlarged side view of the swab of the applicator of FIG. 1.

FIG. 2B is an enlarged view of a collar of the swab of FIG. 2A, taken from portion 2B of FIG. 2A.

FIG. 3 is a perspective view of components that may be used to make up an oral care kit of the present invention, in one embodiment. The components include an applicator, a plurality of swabs held together through one or more support members, and a bottle of topical anesthetic.

FIG. 4A is a perspective view of the applicator of FIG. 1. Here, the sleeve is in its open position, opening the prongs to expose a channel.

FIG. 4B is another perspective view of the applicator of FIG. 1. Here, the sleeve has been moved to its closed position.

FIG. 4C is a cross-sectional view of the applicator of FIG. 1, taken across line 4C-4C of FIG. 4B.

FIG. 4D is another cross-sectional view of the applicator of FIG. 1. Here, the view is taken across line 4D-4D of FIG. 4B.

FIGS. 5A and 5B present alternative arrangements for packaging. In FIG. 5A, a packaging representing a box is shown. In FIG. 5B, a packaging representing a cardboard backing and a plastic cover are shown.

### DETAILED DESCRIPTION

### Definitions

As used herein, the term “swab” means any elongated device that may be used to apply topical anesthetic to an affected area of a patient's mouth.

The term “dental area” refers to the mouth and gums of a patient, including the lips, tongue, teeth and inner cheeks.

### Detailed Description of Certain Embodiments

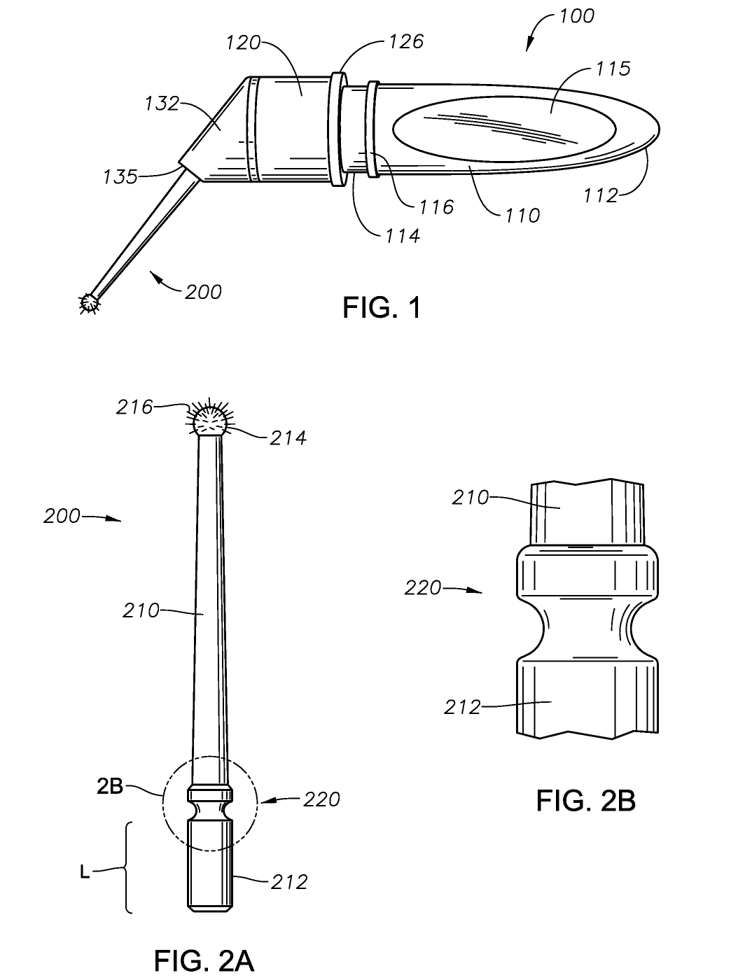

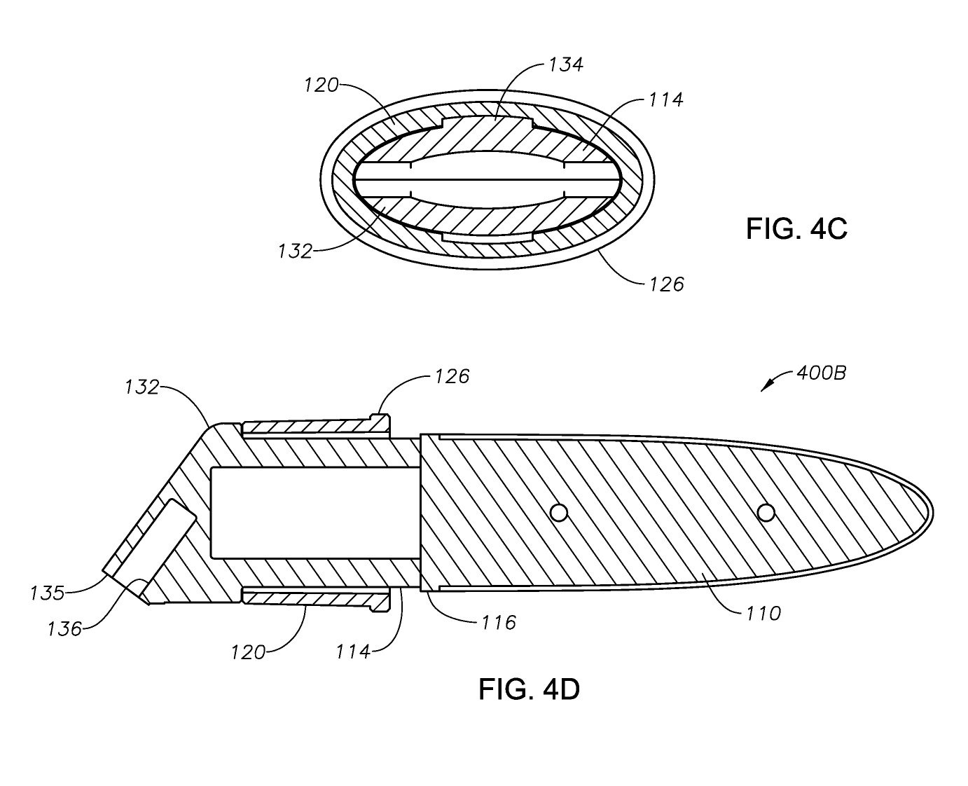

FIG. 1 provides a perspective view of an applicator 100 as may be used in an oral care kit of the present invention, in one embodiment. The applicator 100 first includes a shaft 110. The shaft 110 has a proximal end 112 that serves as a handle. The applicator 100 also includes a distal end 114 that supports a pair of prongs. In the view of FIG. 1, only a front prong 132 is shown. However, front 132 and back 134 prongs are indicated in the perspective views of FIGS. 4A and 4B and in the cross-sectional view of FIGS. 4C and 4D, discussed below.

A sleeve 120 is placed around the shaft 110. The sleeve 120 slides along the distal end 114 of the shaft 114. In the view of FIG. 1, the sleeve 120 is in its closed position. In this position, the prongs 132 and 134 are pulled together to hold a swab 200. As will be described more fully below, the sleeve 120 may be moved to an open position to release the swab 200, and to then receive a new swab. This involves moving the sleeve downward so that a collar 126 of the sleeve 120 lands along a collar 116 along the shaft 110.

The applicator 100 has an optional concave surface portion 115. The concave surface portion 115 resides intermediate the proximal 112 and distal 114 ends. The concave surface portion 115 assists the user in holding the applicator and manipulating the sleeve 116.

As noted, in the view of FIG. 1, a swab 200 extends from the shaft 110. More specifically, the swab 200 extends from a lip 135 of the applicator 100.

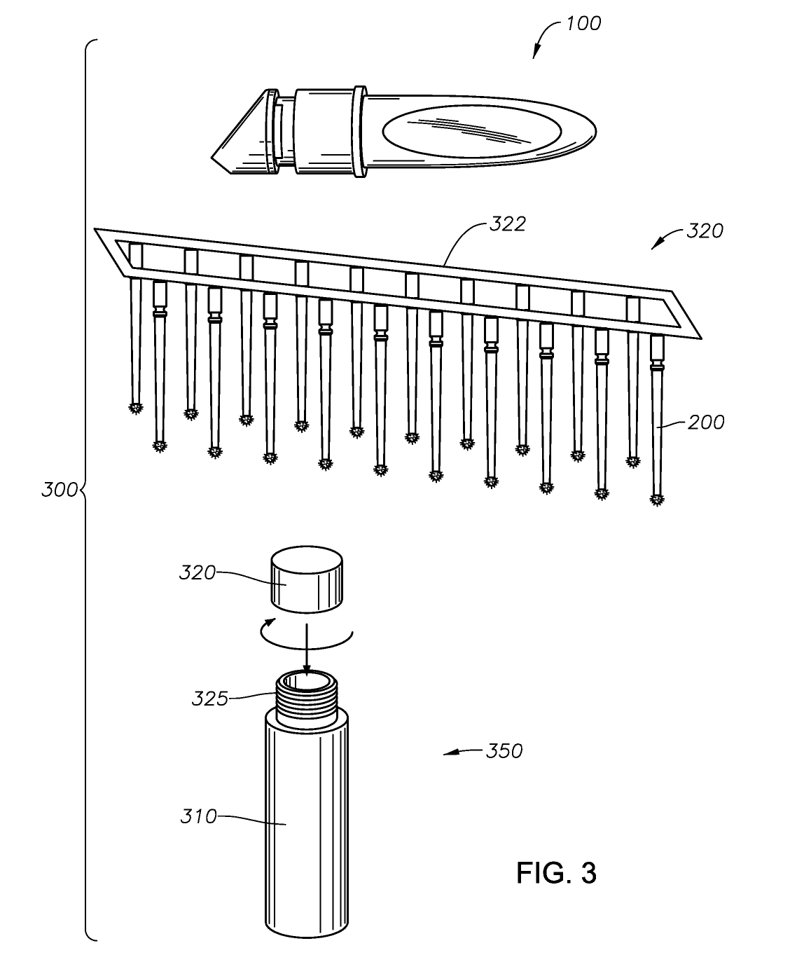

FIG. 2A is an enlarged side view of the swab 200 of the applicator 100 of FIG. 1. It can be seen that the swab 200 defines an elongated body 210 having a proximal end 212 and a distal end 214. Preferably, the body 210 is fabricated from a plastic or an elastomeric material, allowing the body 210 to be flexible and not brittle.

The proximal end 212 of the body 210 is dimensioned to be received between the prongs 132, 134 of the applicator 100. A length “L” is shown indicating a length of the body 210 that is received within the prongs 132, 134. Preferably, a collar 220 is formed along the body 210 near the proximal end 212. The collar 220 provides a catch for the lip 135, helping to maintain the swab 200 within the prongs 132, 134 during use.

FIG. 2B is an enlarged view of the collar 220 of the swab 200 of FIG. 2A. This view is taken from portion 2B of FIG. 2A.

Returning to FIG. 2A, it can be seen that the body 210 also includes a distal end 214. The distal end 214 is preferably rounded, or spherical, to avoid edges. In this respect, the distal end 214 is intended to be introduced into the mouth of a patient to deliver a topical anesthetic. Preferably, the distal end 214 includes a flocking material. Flocking material is shown at 216. The flocking material 216 is a cotton or other cellulosic or open-celled material.

The flocking material 216 is attached to the distal end 214 using a unique manufacturing process. The process involves placing an adhesive on the bulbous distal end. The swab 200 is then placed in an electro-static chamber having flocking material 216 swirling therein. The flocking material 216 attaches to the distal end 214 by action of the adhesive. The fibers of the flocking material 216 are oriented outward from the distal end 214 in response to the electrical charge residing in the chamber.

Various natural fibers are suitable for use as the flocking material 216. Cotton is preferred, but pulped softwood and hardwood fibers may be considered. Absorbent fibrous materials may alternatively be used as derived from Esparto grass, bagasse, jute, ramie, kenaf, sisal, abaca, hemp, oat fiber and flax.

Hydrophilic synthetic fibers may also be considered for the flocking material 216. Suitable synthetic fibers include polyester (including polyethylene terephthalate (PET) and polypropylene), nylon, and the various bi-component fibers known in the art of non-woven substrates. In one aspect, cellulose fibers may be blended with synthetic fibers.

The flocking material 216 may optionally be coated with or otherwise contain an anti-microbial agent. An anti-microbial agent inhibits the risk of spreading infections. Suitable anti-microbial substances include those available from suppliers such as Aegis Environmental Management, Inc. and Microban International, Ltd, as well as others known in the art. Anti-microbial substances may be applied through a variety of methods including, without limitation, using a flexographic printing press and subsequent curing in an oven or by ultraviolet radiation, or simply by spraying the substance onto the absorbent outer surfaces of the fibers during manufacture.

The anti-microbial agent may be, for example, any of the peptides disclosed in U.S. Pat. No. 7,713,927, entitled “Antimicrobial Peptides.” The '927 patent states that the listed peptides are effective in eradicating streptococcus mutans, which are a common oral pathogen and the causative agent of dental caries. The '927 patent is incorporated herein in its entirety by reference.

Other anti-microbial agents may be employed. For example, the anti-microbial agents may include metal particles averaging less than 0.50 microns, wherein the metal is in a base of polysynthetic oils or is applied as part of a colloidal mixture. The metals may be micronized or submicron sized metal that is 99.9% pure and that is comprised of any of the following including: silver, gold, copper, platinum, and/or organo/inorgano complexes of silver, gold, copper, platinum, aluminum oxide, zinc oxide, boron trinitride and metal or metal oxide. Any of such metals are believed to eliminate the growth of bacterial organisms.

The flocking materials may be scented or flavored using, for example, peppermint oil, cinnamon oil, menthol, wintergreen oil, citrus oil, and the like.

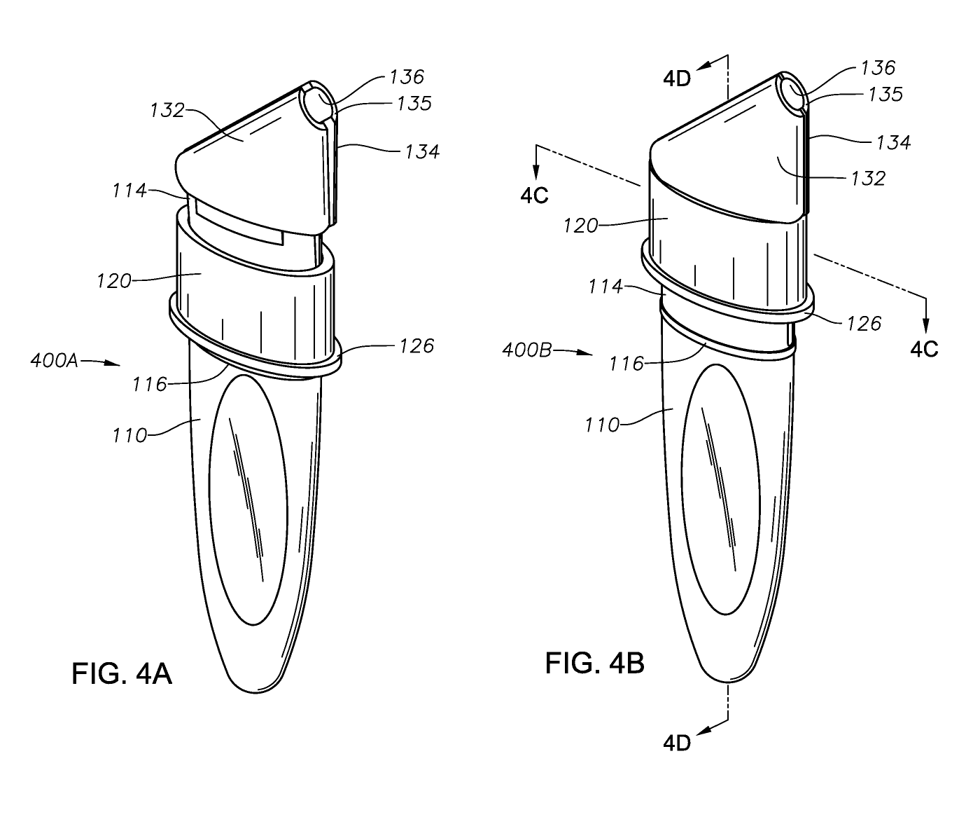

The applicator 100 and a plurality of swabs 200 are sold together as part of a kit. FIG. 3 is a perspective view of components that may be used to make up an oral care kit 300 of the present invention, in one embodiment. According to the view of FIG. 3, the components include an applicator 100, a plurality of swabs 200 joined together as an injection-molded assembly 320, and a bottle of topical anesthetic 350.

In the illustrative arrangement of FIG. 3, the various swabs 200 are releasably held together in the assembly 320 through one or more flexible support members 322. The support members 322 are connected to the swabs 200 during the injection molding process. Swabs 200 may be removed by simply pulling them from the support members 322. Preferably, the kit 300 will include 10 to 50 swabs 200. A selected individual swab 200 may then be connected to the applicator 100 by inserting the proximal end 212 of the swab 200 into a channel 136 formed between the prongs 132, 134.

FIG. 4A is a perspective view of the applicator 100 of FIG. 1, indicated at 400A. Here, the sleeve 120 is in its open position. The sleeve 120 has been moved downward along the shaft 110. This allows the outwardly-biased prongs 132, 134 to open, thereby exposing the channel 136. The sleeve 120 includes a collar 126 that lands along a collar 116 along the shaft 110. The proximal end 212 of a swab 200 may then be inserted into the channel 136.

FIG. 4B is another perspective view of the applicator of FIG. 1, indicated at 400B. Here, the sleeve 120 has been moved upward along the distal end 114 to its closed position. Upward movement of the sleeve 120 enables the channel 136 to closely hold the proximal end 212 of the swab 200 in a friction fit.

FIG. 4C is a cross-sectional view of the applicator of FIG. 1, taken across line 4C-4C of FIG. 4B. FIG. 4D is another cross-sectional view of the applicator of FIG. 1. Here, the view is taken across line 4D-4D of FIG. 4B. The applicator is indicated at 400B. FIGS. 4C and 4D demonstrate the positioning of the prongs 132, 134 at the distal end 114 of the applicator 400B.

Returning to FIG. 3, and as noted, the kit 300 also includes a topical anesthetic 350. The topical anesthetic may be, for example, procaine, lidocaine, prilocaine, benzocaine, or combinations thereof. The topical anesthetic 350 is packaged in a small bottle 310, such as a bottle holding 1.5 fluid ounces. The bottle 310 includes an upper end having male threads 325. The male threads 325 are dimensioned to receive a cap 320 having female threads 320. Optionally, a seal (not shown) is placed over the cap 320 or over the threads 325 to ensure the integrity of the components through the shipping and retail sale process.

The kit 300 may be presented in a box, a blister pack, a cardboard backing encapsulated by plastic, or any other method known in the packaging industry. The kit 300 will include instructions for use and suitable FDA (or other) notices.

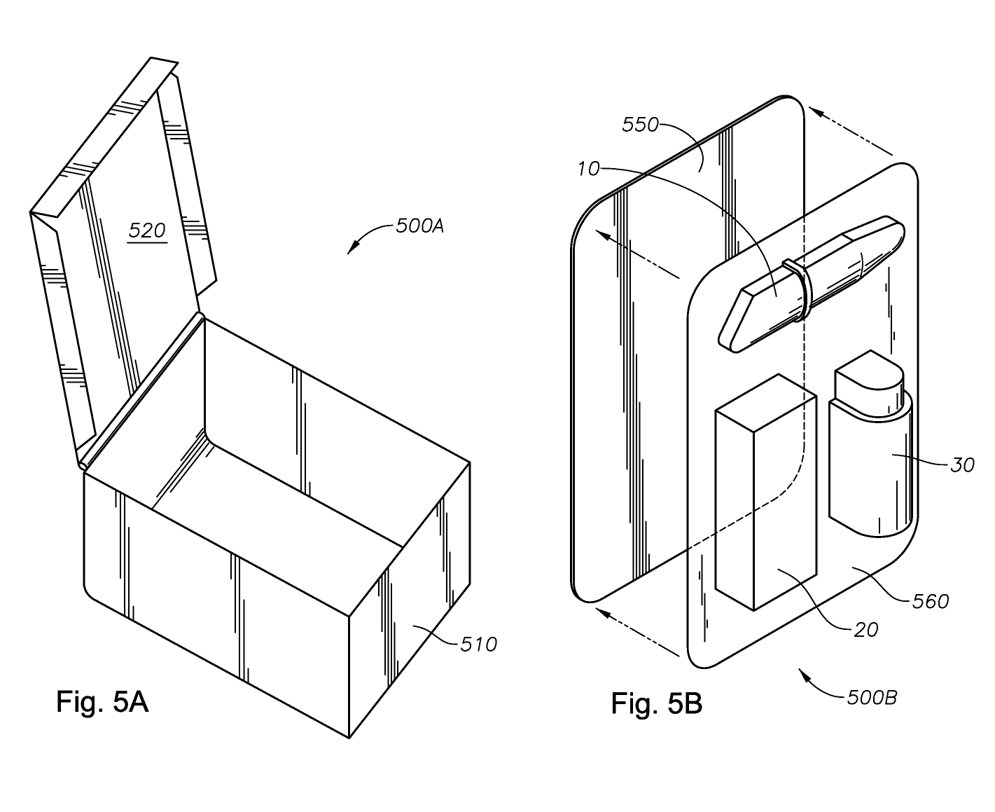

FIGS. 5A and 5B present alternative arrangements for packaging. In FIG. 5A, a packaging 500A representing a box is shown. The box 500A includes side walls 510 and a lid 520. The box 500A is dimensioned to hold at least the applicator 100, an assembly 320 of disposable swabs 200, and a bottle of topical anesthetic 350.

In FIG. 5B, a packaging 500B representing a cardboard backing 550 with a plastic cover 560 are shown. The plastic cover 560 is shown exploded away from the cardboard backing 550 for illustrative purposes. The plastic cover 560 preferably has molded integral recesses 10, 20, 30 for enclosing the applicator 100, an assembly 320 of disposable swabs 200, and a bottle of topical anesthetic 350, respectively.

As can be seen, an improved oral care kit for ameliorating pain in the dental area is provided. Using the kit, a user may apply a topical anesthetic to the mouth of a patient in a quick, convenient and sterile manner. Thus, a method for numbing a dental area is also provided herein.

In one embodiment, the method first includes accessing an applicator. The applicator includes the shaft 110 and the pair of outwardly-biased prongs 132, 134 as described above. The applicator also includes the channel 136 formed along an inner surface of the prongs 132, 134, and a plurality of disposable swabs 200. Further, the applicator includes the sleeve 120 slideably disposed along the distal end 114 of the shaft 120 as described above.

The method also includes positioning the sleeve in its open position. The method then includes sliding the proximal end of a selected swab into the channel, and then moving the sleeve into its closed position. In this way, the selected swab is secured in place within the channel.

The method next includes applying a topical anesthetic to the distal end of the swab. The anesthetic preferably resides within a tube or a bottle. The method then includes unscrewing (or otherwise removing) a lid from the tube or bottle to access the topical anesthetic. In one aspect, the distal end of the swab is dipped into the tube or bottle while the swab is secured by the applicator.

The method also includes applying the topical anesthetic to an affected area of a patient's mouth. This is done by manually extending the swab into the patient's mouth or onto the patients lips or gums to access the affected area. Thereafter, the swab may be released from the prongs by sliding the sleeve back to its open position, abutting a collar along the shaft of the applicator. The selected swab may then be disposed.

In one aspect, the method further includes the steps of:

sliding the sleeve back to its open position, thereby releasing the selected swab from the prongs;

disposing of the selected swab;

selecting a new swab;

sliding the proximal end of the new swab into the channel;

moving the sleeve into its closed position, thereby securing the new swab in place within the channel;

applying topical anesthetic to the distal end of the new swab; and

re-applying the topical anesthetic to the affected area of the patient's mouth.

As can be seen, a novel method of numbing a dental area is provided. While it will be apparent that the inventions herein described are well calculated to achieve the benefits and advantages set forth above, it will be appreciated that the inventions are susceptible to modification, variation and change without departing from the spirit thereof.

I claim:

1. An oral care kit, comprising:
a topical anesthetic; and
an applicator, comprising:
a shaft having a proximal end defining a handle for a user, and a distal end comprising a pair of outwardly-biased prongs;
a channel formed along an inner surface of the prongs;
a plurality of disposable swabs, each swab comprising a proximal end and a distal end, wherein the proximal end of each swab is dimensioned to be slidably received within the channel, and the distal end comprises hydrophilic flocking material; and
a sleeve slideably disposed along the shaft, wherein the sleeve is movable from an open position that allows the prongs to open, thereby exposing the channel for receiving the proximal end of a swab, and a closed position wherein the channel closes around the proximal end of the swab to provide a friction fit; and
wherein each of the disposable swabs is dimensioned to be received within a user's mouth for application of the topical anesthetic.

2. The oral care kit of claim 1, further comprising:
a packaging containing the topical anesthetic, the plurality of disposable swabs, and the applicator for retail sale.

3. The oral care kit of claim 2, wherein the topical anesthetic resides within a tube or a bottle having a removable cap.

4. The oral care kit of claim 3, wherein the topical anesthetic comprises procaine, lidocaine, prilocaine, benzocaine, or combinations thereof.

5. The oral care kit of claim 4, wherein:
the plurality of swabs comprises between about 10 and 50 individual swabs; and
each of the plurality of swabs is fabricated from a plastic material to form a flexible body that is between about 1 inch and 4 inches in length.

6. The oral care kit of claim 5, wherein the plurality of swabs are pre-packaged in generally parallel relation.

7. The oral care kit of claim 2, wherein each swab is removably connected to an injection-molded support member.

8. The oral care kit of claim 2, wherein the hydrophilic material comprises nylon, cotton, or a combination thereof.

9. The oral care kit of claim 8, wherein the hydrophilic material further comprises an anti-microbial agent.

10. The oral care kit of claim 2, wherein:
each of the prongs comprises a lip at a distal end; and
each of the swabs comprises a collar near the proximal end dimensioned to catch on the lip when the sleeve is in its closed position.

11. A method of numbing a dental area, comprising:
accessing an applicator comprising:
a shaft having a proximal end defining a handle, and a distal end comprising a pair of prongs;
a channel formed along an inner surface of the prongs;
a plurality of disposable swabs, each swab comprising a proximal end and a distal end, wherein the proximal end of the swab is dimensioned to be slidably received within the channel, and the distal end comprises hydrophilic flocking material; and
a sleeve slideably disposed along the shaft, wherein the sleeve is movable from an open position that allows the prongs to open, thereby exposing the channel for receiving the proximal end of the swab, and a closed position wherein the channel closes around the proximal end of the swab to provide a friction fit; and
wherein each of the disposable swabs is dimensioned to be received within a user's mouth for application of the topical anesthetic;
positioning the sleeve in its open position;
sliding the proximal end of a selected swab into the channel;
moving the sleeve into its closed position, thereby securing the swab in place within the channel;
applying a topical anesthetic to the distal end of the swab; and
applying the topical anesthetic to an affected area of a patient's mouth.

12. The method of claim 11, wherein:
the applicator, the plurality of swabs, and the topical anesthetic are packaged together for retail sale; and
accessing an applicator comprises removing the applicator from the packaging.

13. The method of claim 11, wherein:
the topical anesthetic resides within a tube or a bottle;
the method further comprises unscrewing a lid from the tube or bottle to access the topical anesthetic; and
applying a topical anesthetic to the distal end of the swab comprises inserting the distal end of the swab into the tube or bottle while the swab is connected to the applicator.

14. The method of claim 11, wherein the topical anesthetic comprises procaine, lidocaine, prilocaine, benzocaine, or combinations thereof.

15. The method of claim 11, wherein:
the plurality of swabs comprises between about 10 and 50 individual swabs; and
each of the plurality of swabs is fabricated from a plastic material to form a flexible body that is between about 1 inch and 4 inches in length.

16. The method of claim 11, wherein:
the plurality of swabs are packaged to be removably connected to an injection-molded support member; and
the method further comprises removing the selected swab from the support member for sliding into the channel.

17. The method of claim 11, further comprising:
sliding the sleeve back to its open position, thereby releasing the selected swabs from the prongs;
disposing of the selected swab;
selecting a new swab;
sliding the proximal end of the new swab into the channel;
moving the sleeve into its closed position, thereby securing the new swab in place within the channel;
applying topical anesthetic to the distal end of the new swab; and
re-applying the topical anesthetic to the affected area of the patient's mouth.

18. The method of claim 11, wherein the hydrophilic material comprises nylon, cotton or a combination thereof.

19. The method of claim 18, wherein the hydrophilic material further comprises an anti-microbial agent.

20. The method of claim 11, wherein the affected area is a tooth, an area of a lip, or an area along a gum line of the patient.
